(19) United States
(12) Patent Application Publication
(10) Pub. No.: US 2005/0251869 A1
(43) Pub. Date: Nov. 10, 2005

(54) Animal model for raising HDL-cholesterol with niacin

(76) Inventors: Tian-Quan Cai, Edison, NJ (US); Melba Hernandez, Clark, NJ (US)

Correspondence Address: MERCK AND CO., INC, P O BOX 2000, RAHWAY, NJ, 07065-0907, US

(21) Appl. No.: 11/121,811

(22) Filed: May 4, 2005

Related U.S. Application Data
(60) Provisional application No. 60/567,986, filed on May 4, 2004.

(51) Int. Cl. A01K 67/027

(52) U.S. Cl. 800/3; 800/18

(57) ABSTRACT

Transgenic mice having human CETP genes are useful models for the action of niacin in humans. Such animals exhibit an increase in HDL-C and the HDL-C/non-HDL-C ratio and a decrease in non-HDL-C when they are treated with niacin. The changes in HDL-C and non-HDL-C appear to be dose-dependent.

### CROSS-REFERENCE TO RELATED APPLICATIONS

This application claims priority under 35 U.S.C. § 119(e) from U.S. provisional application Ser. No. 60/567,986 filed May 4, 2004.

### BACKGROUND OF THE INVENTION

Niacin (nicotinic acid) is a well-known, effective lipid-regulating agent that reduces plasma free fatty acids (FFA), triglycerides (TG), and non-HDL-cholesterol, and increases HDL-cholesterol in humans (1, 2). Niacin is effective in raising HDL-C and decreasing non-HDL-C in humans, but its use is associated with side effects, such as flushing, that have limited its usefulness in treating patients with low HDL-C and high non-HDL-C.

The ability of niacin to reduce plasma free fatty acid (FFA) levels has been well studied. It is believed to proceed through activation of the recently discovered niacin receptor, a Gᵢ-G protein-coupled receptor, termed HM74a in humans and PUMA-G in mice (4-6). The protein is expressed on adipocytes and immune cells, and in humans there is a closely related receptor termed HM74 that appears to have arisen by gene duplication (4-7). Activation of PUMA-G results in a Gᵢ-protein-mediated inhibition of adenylyl cyclase, which results in lower levels of intracellular cyclic adenosine monophosphate (cAMP). In adipose tissue, the ensuing reduced activity of cAMP-dependent protein kinase A results in lower hormone sensitive lipase (HSL) activity, and therefore less hydrolysis of TG to FFA. The consequent reduction in secretion of FFA is thought to deprive the liver of some of the fuel for TG synthesis (34), resulting in lower VLDL and LDL levels. Studies with PUMA-G null mice showed that the nicotinic acid-induced reduction in FFA, as well as TG, requires PUMA-G (4).

The mechanism by which niacin raises HDL-C remains unclear. This is in part due to the fact that although niacin increases HDL-C in humans, it does not increase HDL-C in several common animal models, including the mouse, rat, hamster and dog. It is believed that HDL-C is increased as a result of the interaction of niacin with the niacin receptor, but this has not been proved at this time. Animal models are available for studying the effects of niacin and compounds that act on the niacin receptor to reduce FFA, TG and non-HDL cholesterol, including the mouse, rat, hamster and dog. These animals are not suitable for measuring the HDL-raising effects of niacin. Searches have been carried out to find a reliable animal model for the HDL-raising effect of niacin, but these searches have so far been unsuccessful. Pigs and rabbits have been suggested as models for the potential HDL-raising effects of niacin. However, due to the larger sizes of these model animals and the need for a longer treatment time to obtain reliable data (>10 weeks of treatment), the usefulness of pigs or rabbits as tools for studying the effects of niacin or for drug discovery has been limited.

Cholesterol ester transfer protein (CETP) plays an important role in HDL-metabolism in humans. Inhibition of the action of CETP in humans raises the ratio of HDL to non-HDL-cholesterol. A number of species, including the mouse, that lack the CETP gene, have a significantly higher ratio of HDL to non-HDL-cholesterol compared with animals that have the CETP gene. A recent study (8) has shown that transgenic mice with an over-expression of the human CETP gene have a lipoprotein profile that is more like the human lipoprotein profile in comparison with the lipoprotein profile in wild type mice. The HDL to non-HDL ratio is significantly lower in transgenic mice having the human CETP gene compared with mice that do not have the CETP gene.

A reliable animal model for evaluating the effects of niacin and compounds that interact with the niacin receptor would be a valuable tool for understanding the action of niacin in raising HDL-C and for improving niacin formulations. Such a model would also be a useful tool for selecting and evaluating new compounds that have effects similar to those of niacin, such as compounds that have been identified as ligands of the niacin receptor (e.g. agonists). Such a model may be useful for identifying new compounds that raise HDL-C, reduce non-HDL-C, including VLDL-C and LDL-C, and reduce TG's, either through interactions with the niacin receptor or through a mechanism that is yet to be elucidated. These new compounds may be useful in human drug formulations for treating, preventing and/or reducing the risk of developing atherosclerosis. (8-10)

### SUMMARY OF THE INVENTION

Transgenic mammals, which may be mice or rats, that have human CETP genes are useful models for the action of niacin in humans and mammals. Such animals exhibit an increase in HDL-C and the HDL-C/non-HDL-C ratio and a decrease in non-HDL-C when they are treated with niacin. The changes in HDL-C and non-HDL-C appear to be dose-dependent.

### DETAILED DESCRIPTION OF THE INVENTION

Transgenic mammals, which may be mice or rats, that have human CETP genes, may be used to test compounds for niacin-like activity. The method of testing a compound for niacin-like activity comprises the administration of a test compound to a transgenic mouse or rat, wherein the mammal comprises a human CETP gene in its genome, and measuring the change in the amount of HDL-C in the transgenic mouse or rat after administration of the test compound for a period of time sufficient to effect a change in HDL-C and in a quantity sufficient to effect such a change.

Preferred CETP genes are human CETP genes. Preferred test animals are mice. Transgenic mice having human CETP as part of their genome are known and are commercially available (see Examples). Methods of making transgenic species are well known, and transgenic mice having different portions of human CETP in their genome can be made using methods well known to practitioners in the fields of biology and biotechnology. Such transgenic mice can also be made with other genes also included in their genome. For example, mice having human CETP genes and one or more other human genes were used in the examples, where the second gene was for example the gene that causes expression of apolipoprotein B 100 (apoB100) or apoA1. The DNA that is used in making transgenic mice can also be DNA that selectively causes expression of CETP, apoB 100, and/or apoA1 in a target organ, such as the liver, as was the case with the transgenic mice that were used herein. The DNA fragments that are used in making the genes of the transgenic mice are in general long enough and in sufficient quantity to cause expression of CETP protein in the transgenic mouse, and particularly in the target organ of the mouse (i.e. the liver). Transgenic mice having human CETP, optionally with other genes, in their genome can also be purchased (see examples).

In carrying out the tests for compounds having niacin-like activity, the compounds are administered for a time period long enough to effect a change in HDL-C. The time period depends on many factors, and may be less than a week (e.g. 4-5 days), but longer time periods may be necessary to obtain reliable measurements of changes in HDL-C, as for example 1 week, 2 weeks, 3 weeks, or 4 weeks.

Compounds that have niacin-like activity are ligands for the niacin receptor, and generally are agonists of the niacin receptor. Such compounds will generally cause a reduction of non-HDL-C, such as LDL-C and VLDL-C, a reduction of triglycerides (TG), and an increase in HDL-C. A compound that is identified as having niacin-like activity by the method of this invention will cause an increase in the amount of HDL-C in the transgenic mouse of at least 10% compared with the amount of HDL-C that is observed in an untreated transgenic mouse used as a control. The compound may cause a larger increase in HDL-C, on the order of 25%, 40%, or 50% or more. The quantity of test compound required to bring about an increase in HDL-C as described above will vary depending on several factors, such as the potency of the compound and its bioavailablility. A compound identified as having niacin-like activity based on the elevation of HDL-C as described above would be considered a candidate for further evaluation as a potential new drug in accordance with procedures that are well known to practitioners in drug development in the pharmaceutical industry.

Finally, the invention also includes a kit which comprises a transgenic mouse which has the human CETP gene in its genome sufficient to express CETP protein, a device for collecting one or more samples of blood or serum from the mouse for measurement of HDL-C, and one or more compounds that are to be tested for niacin-like activity, where the test compounds are niacin receptor ligands, in particular niacin receptor agonists.

### REFERENCES

1. Knopp R H. Drug treatment of lipid disorders. N Engl J Med. 34:498-511, 1999.
2. Szapary P O and Rader D J. Pharmacological management of high triglycerides and low high-density lipoprotein cholesterol. Curr Opin Pharmacol. 1: 113-20, 2001.
3. Carlson L A. Studies on the effect of nicotinic acid on catecholarnine stimulated lipolysis in adipose tissue in vitro. Acta Med Scand. 173:719-22, 1973.
4. Tunaru S, Kero J, Schaub A, Wufka C, Blaukat A, Pfeffer K, Offermanns S. PUMA-G and HM74 are receptors for nicotinic acid and mediate its anti-lipolytic effect. Nat Med. 9:352-5, 2003.
5. Wise A, Foord S M, Fraser N J, Barnes A A, Elshourbagy N, Eilert M, Ignar D M, Murdock P R, Steplewski K, Green A, Brown A J, Dowell S J, Szekeres P G, Hassall D G, Marshall F H, Wilson S, Pike N B. Molecular identification of high and low affinity receptors for nicotinic acid. J Biol Chem. 278:9869-74, 2003.
6. Soga T, Kamohara M, Takasaki J, Matsumoto S, Saito T, Ohishi T, Hiyama H, Matsuo A, Matsushime H, Furuichi K. Molecular identification of nicotinic acid receptor. Biochem Biophys Res Commun. 303:364-9, 2003.
7. Schaub A, Futterer A, Pfeffer K. PUMA-G, an IFN-gamma-inducible gene in macrophages is a novel member of the seven transmembrane spanning receptor superfamily. Eur J Immunol. 31:3714-25, 2001.
8. Agellon L B, Walsh A, Hayek T, Moulin P, Jiang X C, Shelanski S A, Breslow J L, Tall A R. Reduced high density lipoprotein cholesterol in human cholesteryl ester transfer protein transgenic mice. J Biol Chem. 266:10796-801, 1991.
9. Linton M F, Farese R V Jr, Chiesa G, Grass D S, Chin P, Hammer R E, Hobbs H H, Young S G. Transgenic mice expressing high plasma concentrations of human apolipoprotein B100 and lipoprotein(a). J Clin Invest. 92:3029-37, 1993.
10. Grass DS, Saini U, Felkner R H, Wallace R E, Lago W J, Young S G, Swanson M E. Transgenic mice expressing both human apolipoprotein B and human CETP have a lipoprotein cholesterol distribution similar to that of normolipidemic humans. J Lipid Res. 36:1082-91, 1995.

### EXAMPLES

Materials: The mice used in these studies were pathogen-free C57BI/6J mice of the following genotypes: (1) wild type; (2) hemizygous human CETP-transgenic (hCETP-tg); (3) hemizygous human apoB100-transgenic, and 4) hemizygous human CETP and apoB100 dual-transgenic mice. The transgenic mice were purchased from Taconic Laboratories, Germantown, N.Y., where they were generated by introduction of genomic DNA fragments that drive expression of human CETP and/or apoB100 in the mouse liver (8-10).

In addition, hemizygous human-CETP/human-apolipoprotein A1 dual-transgenic mice were generated by cross-breeding homozygous human-CETP transgenic male mice, purchased from Taconic, with homozygous human-apolipoprotein A1 transgenic female mice, purchased from Jackson Laboratories.

### Example 1

Methods: Male mice (hCETP-tg and wild type C57BI/6J control mice; ˜10 weeks of age; 5-7/group) were used in this study. The mice were fed a normal rodent chow diet (Purina 7012) along with varying levels of niacin (milled with diet, expressed as % by weight of diet). They were housed with normal lighting and were allowed access to food and water ad lib. Blood samples were obtained by retro-orbital plexus at one, two and three weeks of treatment. After the fourth week of treatment, the mice were fasted for four hours and then euthanized. After euthanization, blood samples were collected by cardiac puncture. Serum lipids (total cholesterol, HDL-cholesterol, TG and FFA, and lipoprotein profiles) were determined. Serum samples from four weeks of treatment were also measured for CETP enzyme activity.

Results: The hCETP-tg mice that were not treated with niacin exhibited significantly lower HDL-cholesterol levels than the wild-type (WT) control mice that were not treated with niacin (˜40%, data not shown). Treatment of wild-type control mice with niacin resulted in a broad reduction of serum lipids, including TG, total cholesterol, HDL-C, non-HDL-C, and FFA. However, treatment of hCETP-tg mice with niacin resulted in 1) an increase of HDL-cholesterol, and 2) a reduction of non-HDL cholesterol, TG and FFA. These changes appear to be dose-dependent. For the WT mice that were treated with 1% niacin, the changes in HDL-C at weeks 14 compared with untreated WT mice were: 1 week, −9%; 2 weeks, −17%; 3 weeks, −13%; 4 weeks, −7%. For the h-CETP-tg mice that were treated with 1% niacin, the changes in HDL-C at weeks 1-4 compared with untreated h-CETP-tg mice were: 1 week, +40%; 2 weeks, +38%; 3 weeks, +37%; 4 weeks, +23%. Analysis of lipoprotein profiles by size exclusion chromatography revealed a rise of cholesterol in the HDL fractions, and a decline of TG in the VLDL fractions. While these effects appeared to be evident throughout the study, the overall effects at week four were not as dramatic as the effects at earlier time points. It is unclear whether this is due to a longer treatment time or is due to the difference in bleeding route (orbital vs cardiac).

### Example 2

Methods: This two-week study is similar to the four-week study described in Example 1, except as noted below. Blood samples were obtained by retro-orbital plexus after one week of treatment, as in Example 1. After two weeks of treatment, the mice were fasted for four hours and then euthanized. Blood samples were collected by cardiac puncture. Serum lipids (total cholesterol, HDL-cholesterol, total triglyceride and FFA) were determined.

Results: Niacin treatment resulted in a reduction of serum TG and an increase of HDL-C. At one week of treatment, 1% niacin led to a 44% reduction of TG and a 35% increase of HDL-C. At two weeks of treatment, 1% niacin led to a 45% reduction of TG and a 37% increase of HDL-C. Analysis of lipoprotein profiles by size exclusion chromatography revealed an increase of cholesterol content in the HDL particles without significant change of the particle size. The cholesterol content of the LDL and VLDL particles were reduced. TG contents of VLDL, LDL and HDL particles were all reduced.

### Example 3

Examples 1 and 2 illustrate that transgenic mice carrying the human CETP gene have a reduced ratio of HDL-C/non-HDL-C compared with WT mice, and that niacin raises the HDL-C level and the ratio of HDL-C/non-HDL-C ratio in the transgenic mice. Experiments similar to those in Examples 1 and 2 were carried out using human ApoB 100 transgenic mice. The untreated human-ApoB 100 transgenic mice have a more human-like lipoprotein profile, including a lower amount of HDL-C than is found in wild-type mice. In these studies, niacin does not raise the level of HDL-C as it does in the h-CETP-tg mice.

### Example 4

Methods: This example is a study of the lipid modulating effect of niacin in human CETP and Apo-B100 dual transgenic mice. These mice have a more human-like lipoprotein profile than WT mice, with a lower HDUnon-HDL-C ratio.

Mice (hemizygous human-CETP/ApoB100 dual transgenic mice, ˜10 weeks of age, 7-8/group) were fed a normal rodent chow diet (Purina 7012) along with varying levels of niacin (milled with the feed). They were housed with normal lighting and were allowed access to food and water ad lib. Blood samples were obtained by retro-orbital plexus after one week of treatment. After two weeks of treatment, the mice were fasted for four hours and then euthanized. Blood samples were collected by cardiac puncture. Serum lipids (total cholesterol, HDL-cholesterol, TG and FFA, and lipoprotein profiles) were determined.

Results: The untreated human-CETP/ApoB 100 dual-transgenic mice have significantly higher levels of LDL-C and much lower levels of HDL-C when compared with those of regular mice. One week of treatment with niacin resulted in a decrease of TC and TG without a significant change in the amount of HDL-C. After 2 weeks of treatment, however, a significant increase of HDL-C was observed in animals that were fed 0.1% and 1% niacin in their diet. There was a 41% increase in HDL-C in animals that were fed 0.1% niacin for two weeks; a 5% decrease in HDL-C in animals that were fed 0.3% niacin for two weeks; and a 15% increase in HDL-C in animals that were fed 1% niacin for two weeks. Analyses of lipoprotein profiles by size exclusion chromatography for samples that were taken after two weeks of treatment showed an increase of cholesterol in the HDL fractions, a decrease of cholesterol in the LDL fractions, and a decrease of TG in the VLDL and LDL fractions.

### Example 5

Methods: Mice used in this study express both human CETP and human apolipoprotein A1 genes. The mice were generated by cross-breeding homozygous human-CETP transgenic male mice with homozygous female human-apolipoprotein A1-transgenic mice. Female mice (˜10 weeks of age, 10/group) were fed a normal rodent chow diet (Purina 7012) with or without 1% niacin (milled with the feed). They were housed with normal lighting and were allowed access to food and water ad lib. After seven days of treatment, the mice were fasted for four hours and then euthanized. Blood samples were collected by cardiac puncture. Serum lipids (total cholesterol, HDL-cholesterol, TG and FFA, and lipoprotein profiles) were determined.

Results: Treatment of the female human-CETP/human-apoA1 dual-transgenic mice with 1% niacin for one week resulted in a significant elevation of HDL-C (˜38%) and a reduction of TG (53%) and non-HDL-C (39%). Treatment of the male human-CETP/human-apoA1 dual-transgenic mice with 1% niacin for one week resulted in a significant elevation of HDL-C (27%) and a reduction of TG (36%). Non-HDL-C did not change significantly.

1. A method of testing a compound for niacin-like activity in raising HDL-C in a human patient comprising the administration of a test compound which is a niacin receptor ligand to a transgenic mammal selected from the group consisting of a transgenic mouse and transgenic rat, said transgenic mammal comprising a human CETP gene in its genome, and measuring the change in the amount of HDL-C in said transgenic mammal after administration of the test compound for a period of time sufficient to effect a change in HDL-C.

2. The method of claim 1, wherein said transgenic mammal is a transgenic mouse, and said test compound is a niacin receptor agonist.

3. The method of claim 2, wherein said test compound is administered for a time period of at least one week.

4. The method of claim 3, wherein said test compound increases the amount of HDL-C in the serum or blood of said transgenic mouse by at least 10% compared with the amount of HDL-C in the serum or blood of an untreated mouse.

5. The method of claim 3, wherein said test compound increases the amount of HDL-C in the serum or blood of said transgenic mouse by at least 25% compared with the amount of HDL-C in the serum or blood of an untreated mouse.

6. A compound having niacin-like activity, wherein said compound is tested using the method of claim 3 and increases the amount of HDL-C in the serum or blood of said transgenic mouse by at least 10% compared with the amount of HDL-C in the serum or blood of an untreated mouse.

7. A compound of claim 6, wherein said compound increases the amount of HDL-C in the serum or blood of said transgenic mouse by at least 25% compared with the amount of HDL-C in the serum or blood of an untreated mouse.

8. The method of claim 3, wherein said test compound is administered for a time period of at least two weeks.
